(19) United States
(12) Patent Application Publication
(10) Pub. No.: US 2019/0292250 A1
(43) Pub. Date: Sep. 26, 2019

(54) AAV-ANTI-VEGF FOR TREATING CANCER IN COMPANION ANIMALS

(71) Applicant: The Trustees of the University of Pennsylvania, Phildelphia, PA (US)

(72) Inventors: Christian Hinderer, Philadelphia, PA (US); James M. Wilson, Philadelphia, PA (US); Matthew Wilson, Philadelphia, PA (US)

(21) Appl. No.: 15/754,939

(22) PCT Filed: Aug. 30, 2016
(86) PCT No.: PCT/US16/49492

§ 371 Date: Feb. 23, 2018

Related U.S. Application Data
(60) Provisional application No. 62/212,170, filed on Aug. 31, 2015.

(51) Int. Cl. C07K 16/22; C12N 15/86

(52) U.S. Cl. CPC C07K 16/22; C12N 15/86; C07K 2319/92; C12N 2840/203; C07K 2317/24; C12N 2750/14143; C12N 2800/22; C07K 2319/50

(57) ABSTRACT

Compositions and methods are provided for treating hemangiosarcoma in a subject. A recombinant AAV comprising a capsid and a vector genome comprising a nucleic acid expression cassette is provided. The expression cassette includes sequences encoding a promoter, a first signal peptide operably linked to an anti-VEGF antibody heavy chain immunoglobulin, a linker sequence, and a second signal peptide operably linked to an anti-VEGF light chain immunoglobulin, wherein said expression cassette co-expresses the immunoglobulin chains in a host cell under conditions which permit the chains to assemble into a functional anti-VEGF antibody. In one embodiment, the anti-VEGF antibody is a chimeric antibody.

### INCORPORATION-BY-REFERENCE OF MATERIAL SUBMITTED IN ELECTRONIC FORM

Applicant hereby incorporates by reference the Sequence Listing material filed in electronic form herewith. This file is labeled “15-7473 SEQ LISTING ST25”.

### BACKGROUND OF THE INVENTION

Hemangiosarcoma (HSA) affects over 250,000 dogs a year in the United States. HSA is a vascular tumor usually located in the spleen or liver. Currently HSA cannot be cured and standard therapy is typically surgery followed by a chemotherapeutic regimen of doxorubicin. This therapy will typically cost $6,000+ and may extend survival for an additional 3-4 months. Although dogs of any age and breed are susceptible to hemangiosarcoma, it occurs more commonly in dogs beyond middle age (older than 6 years), and in breeds such as Golden Retrievers, German Shepherd Dogs, Portuguese Water Dogs, Bernese Mountain Dogs, Flat Coated Retrievers, Boxers and Skye Terriers, among others. According to the Golden Retriever Health Study published in 2000, the estimated lifetime risk of hemangiosarcoma in this breed is 1 in 5, illustrating the magnitude of this problem.

In dogs, the common primary sites for hemangiosarcoma are the spleen, the right atrium of the heart, and the subcutis, which is the tissue beneath the skin. The pattern of growth for these tumors involves infiltration into normal tissues surrounding the tumor as well as metastasis.

HSA tumors also express vascular endothelial growth factor (VEGF) and VEGF receptors that help the tumor to vascularize and proliferate through angiogenic growth. There is no VEGF targeted therapy currently available for dogs or other companion animals.

Adeno associated virus (AAV) is a desirable vector for delivering therapeutic genes due to its safety profile and capability of long term gene expression in vivo. Recombinant AAV vectors (rAAV) have been previously used to express single chain and full length antibodies in vivo. Due to the limited transgene packaging capacity of AAV, it has been a technical challenge to have a tightly regulated system to express heavy and light chains of an antibody using a single AAV vector in order to generate full length antibodies.

Therefore, compositions useful for targeting VEGF in subjects, particularly companion animals, are needed.

### SUMMARY OF THE INVENTION

Novel engineered chimeric canine anti-VEGF antibody constructs are provided. These constructs can be delivered to subjects in need thereof via a number of routes, and particularly by expression in vivo mediated by a recombinant vector such as a recombinant adeno-associated virus (rAAV) vector. In one embodiment, the subject is a companion animal, e.g., a dog or a cat.

In one aspect, a viral vector is provided. In one embodiment, the viral vector includes at least one nucleic acid expression cassette comprising a sequence encoding a chimeric canine vascular endothelial growth factor (VEGF) antibody operably linked to expression control sequences that direct expression of the VEGF antibody in a host cell.

In another embodiment, the viral vector includes at least one nucleic acid expression cassette comprising sequences which encode: a promoter, a first signal peptide operably linked to a chimeric canine anti-VEGF antibody heavy chain immunoglobulin, a linker sequence, and a second signal peptide operably linked to a chimeric canine anti-VEGF light chain immunoglobulin, wherein said expression cassette co-expresses the immunoglobulin chains in a host cell under conditions which permit the chains to assemble into a functional chimeric canine anti-VEGF antibody.

In another aspect, a viral vector comprising at least one nucleic acid expression cassette comprising a nucleic acid sequence encoding a functional anti-VEGF antibody which binds canine VEGF which comprises an anti-VEGF antibody heavy chain immunoglobulin of SEQ ID NO: 15 and/or an anti-VEGF antibody light chain immunoglobulin of SEQ ID NO: 14, and expression control sequences which direct expression of the immunoglobulin chains in a host cell under conditions which permit the chains to assemble into the functional antibody.

In another embodiment, the viral vector includes at least one nucleic acid expression cassette comprising sequences which encode: a 5′ AAV inverted terminal repeat sequence (ITR), a promoter with optional enhancer, a first signal peptide operably linked to a chimeric canine anti-VEGF antibody heavy chain immunoglobulin, a linker sequence, a second signal peptide operably linked to an anti-VEGF light chain immunoglobulin and a 3′ AAV ITR, wherein said expression cassette co-expresses the immunoglobulin chains in a host cell under conditions which permit the chains to assemble into a functional canine chimeric anti-VEGF antibody. Also provided are the chimeric anti-VEGF antibodies produced from the viral vectors. In one embodiment, the anti-VEGF antibody is a chimeric antibody where murine variable chains are linked to canine constant domains. In one embodiment, the viral vector is an adeno-associated viral vector. In another embodiment, the vector is a rAAV having a capsid selected from AAV8, rh64R1, AAV9, AAVhu.37, or rh10 and variants thereof. In one embodiment, the capsid is an AAV8 capsid or a variant thereof.

In another embodiment, the viral vector is selected from another viral vector. Other suitable vectors include, without limitation, adenoviruses, an RNA vector (e.g., retroviruses such as, for example, Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend, Murine Stem Cell Virus (MSCV) and Rous Sarcoma Virus (RSV)). “Retroviral vectors” used in the invention can also include vectors derived from human T cell leukemia viruses, HTLV-1 and HTLV-2, and the lentiviral family of retroviruses, such as human Immunodeficiency viruses, HIV-1, HIV-2, simian immunodeficiency virus (SIV), feline immonodeficiency virus (Hy), equine immunodeficiency virus (Hy), and other classes of retroviruses, a liposome, a cationic lipid, a lentiviral vector, and a transposon. In another embodiment, the viral vector is the vesicular stomatitis virus.

In another aspect, a pharmaceutical composition is provided which includes a pharmaceutically acceptable carrier and a viral vector as described herein. In another embodiment, the pharmaceutical composition is a suspension which includes a viral vector and a carrier, diluent, excipient and/or adjuvant.

In another aspect, a method for treating cancer, e.g., hemangiosarcoma, is provided. In one embodiment, the method includes administering a viral vector-containing composition as described herein.

In another aspect, a chimeric anti-VEGF antibody is provided. In one embodiment, the chimeric antibody includes murine and canine immunoglobulin domains. In another aspect, pharmaceutical compositions which include a chimeric anti-VEGF antibody in combination with a pharmaceutically acceptable carrier, are provided. In another embodiment, the pharmaceutical composition includes a chimeric canine anti-VEGF antibody and a carrier, diluent, excipient and/or adjuvant.

Other aspects and advantages of the invention will be readily apparent from the following detailed description of the invention.

### BRIEF DESCRIPTION OF THE DRAWINGS

FIG. 1 is a photograph of SDS-PAGE showing purified chimeric murine-canine anti-VEGF antibody. The ladder is Pageruler Prestained (Thermo Scientific).

FIG. 2 is a graph showing serum anti-VEGF antibody concentration in 3 dogs treated with an AAV8 vector.

FIG. 3 is a cartoon map of the expression construct used in Example 1.

FIG. 4 is a cartoon map showing the layout of the expression cassette of the construct of FIG. 3 and Example 1.

FIG. 5 shows the light chain (top; SEQ ID NO: 14) and heavy chain (bottom; SEQ ID NO: 15) of a chimeric anti-VEGF antibody of one embodiment of the invention. Complementarity determining regions (CDRs) are underlined.

### DETAILED DESCRIPTION OF THE INVENTION

Described herein are viral vectors which include at least one nucleic acid expression cassette comprising sequences that encode a chimeric anti-VEGF vascular endothelial growth factor (VEGF) antibody operably linked to expression control sequences that direct expression of the VEGF antibody in a host cell. Also provided are chimeric anti-VEGF antibodies containing both canine and murine regions, which have been produced using the constructs described herein. Using an illustrative construct expressed by an AAV8 vector, the inventors have demonstrated sustained antibody expression of 110+ days in in vivo studies.

In one embodiment, the viral vector includes at least one nucleic acid expression cassette comprising nucleic acid sequences which encode: a promoter, a first signal peptide operably linked to a chimeric canine anti-VEGF antibody heavy chain immunoglobulin, a linker sequence, and a second signal peptide operably linked to a chimeric canine anti-VEGF light chain immunoglobulin, wherein said expression cassette co-expresses the immunoglobulin chains in a host cell under conditions which permit the chains to assemble into a functional chimeric anti-VEGF antibody (FIG. 4). In another embodiment, the viral vector includes at least one nucleic acid expression cassette comprising nucleic acid sequences which encode: a 5′ AAV inverted terminal repeat sequence (ITR), a promoter with optional enhancer, a first signal peptide operably linked to a chimeric canine anti-VEGF antibody heavy chain immunoglobulin, a linker sequence, a second signal peptide operably linked to a chimeric canine anti-VEGF light chain immunoglobulin and a 3′ AAV ITR, wherein said expression cassette co-expresses the chimeric canine immunoglobulin chains in a host cell under conditions which permit the chains to assemble into a functional anti-VEGF antibody. As used herein a “functional antibody” may be an antibody or immunoglobulin which binds to a selected target (e.g., VEGF) with sufficient binding affinity to effect a desired physiologic result, which may be protective (e.g., passive immunization) or therapeutic (e.g., neutralizing VEGF).

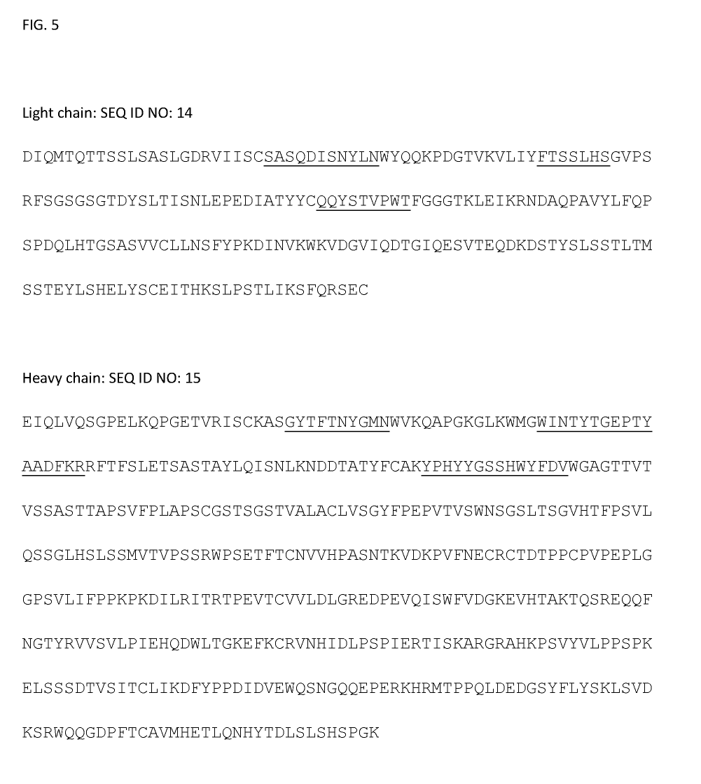

In one embodiment, the anti-VEGF antibody is a chimera. As used herein, a “chimera” refers to an antibody which incorporates regions from proteins from two or more species, to impart properties from each of the “parent” proteins to the resulting chimeric antibody. In one embodiment, the antibody contains murine immunoglobulin domains. In one embodiment, the antibody contains canine immunoglobulin domains. In one embodiment, the antibody contains murine variable regions. In another embodiment, the antibody contains variable chain regions from another species. In one embodiment, the antibody contains variable regions from murine antibody and constant chain regions from a canine, and the resulting antibody is referred to as “chimeric”. In another embodiment, the antibody contains constant chain regions from the same subject species for which administration of the antibody is ultimately intended. In one embodiment, the Fc regions are canine sequences. In another embodiment, the anti-VEGF antibody comprises the variable regions of a murine anti-VEGF antibody and canine VEGF IgGA/kappa constant regions. In one embodiment, the murine variable regions are from murine monoclonal antibody a4.6.1 (see Gerber et al, Mice expressing a humanized form of VEGF-A may provide insights into safety and efficacy of anti-VEGF antibodies, PNAS, 104(9):3478-83 2007, which is incorporated by reference). In one embodiment, the antibody comprises the sequences of SEQ ID NO: 14 and SEQ ID NO: 15. In another embodiment, the antibody is a chimeric canine antibody comprising the murine CDRs shown in FIG. 5. In this embodiment, the remaining antibody sequences are from a canine.

The AAV vector provided herein may contain 1 or 2 open reading frames (ORF) expressing one or more immunoglobulin domains. As used herein, an “immunoglobulin domain” refers to a domain of an antibody heavy chain or light chain as defined with reference to a conventional, full-length antibody. More particularly, a full-length antibody contains a heavy (H) chain polypeptide which contains four domains: one N-terminal variable (VH) region and three C-terminal constant (CH1, CH2 and CH3) regions and a light (L) chain polypeptide which contains two domains: one N-terminal variable (VL) region and one C-terminal constant (CL) region. A Fab region may contain one constant and one variable domain for each the heavy and light chains.

The term “immunoglobulin” is used herein to include antibodies, and functional fragments thereof, including immunoglobulin domains, as described above. Anti-VEGF antibodies as described herein may exist in a variety of forms including, for example, polyclonal antibodies, monoclonal antibodies, camelized single domain antibodies, intracellular antibodies (“intrabodies”), recombinant antibodies, multispecific antibody (bispecific), antibody fragments, such as, Fv, Fab, F(ab)₂, F(ab)₃, Fab′, Fab′-SH, F(ab′)₂, an immunoadhesion, single chain variable fragment antibodies (scFv), tandem/bis-scFv, Fc, pFc′, scFvFc (or scFv-Fc), disulfide Fv (dsfv), bispecific antibodies (bc-scFv) such as BiTE antibodies; camelid antibodies, resurfaced antibodies, humanized antibodies, fully human antibodies, caninized antibodies, fully canine antibodies, single-domain antibody (sdAb, also known as NANOBODY®), chimeric antibodies, chimeric antibodies comprising at least one canine constant region, and the like. “Antibody fragment” refers to at least a portion of the variable region of the immunoglobulin that binds to its target, e.g., VEGF. In one embodiment, the immunoglobulin is an IgG. However, other types of immunoglobulin may be selected.

In one embodiment, the anti-VEGF antibody is a chimeric monoclonal antibody. In one embodiment, the anti-VEGF antibody heavy chain immunoglobulin includes a variable chain sequence (VH). In one embodiment, the anti-VEGF antibody heavy chain immunoglobulin includes a variable chain sequence (VH) and at least one canine constant chain sequence (CH). In a further embodiment, the anti-VEGF antibody heavy chain immunoglobulin includes a variable chain sequence (VH) and all three constant chain sequences (CHL CH2 and CH3). In one embodiment, the anti-VEGF antibody variable heavy chain immunoglobulin amino acid sequence is SEQ ID NO: 1. In one embodiment, the constant heavy chain amino acid sequence is SEQ ID NO: 2. In one embodiment, the anti-VEGF light chain immunoglobulin includes a variable chain sequence (VL). In one embodiment, the anti-VEGF light chain immunoglobulin includes a variable chain sequence (VL) and at least one canine constant chain sequence (CL). In one embodiment, the anti-VEGF light chain variable immunoglobulin amino acid sequence is SEQ ID NO: 3. In one embodiment, the anti-VEGF light chain constant immunoglobulin amino acid sequence is SEQ ID NO: 4. In one embodiment, the chimeric anti-VEGF light chain sequence is SEQ ID NO: 14. In one embodiment, the chimeric anti-VEGF heavy chain sequence is SEQ ID NO: 15.

The term “heterologous” when used with reference to a protein or a nucleic acid indicates that the protein or the nucleic acid comprises two or more sequences or subsequences which are not found in the same relationship to each other in nature. For instance, the expression cassette is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the immunoglobulin coding sequences, the promoter is heterologous. Likewise, with reference to the AAV capsid, the immunoglobulin coding sequence is heterologous.

The one or more ORF(s) carried by the nucleic acid molecule packaged within the vector may be expressed from two expression cassettes, one or both of which may be bicistronic. Thus, when referring to an expression cassette, or a vector comprising an expression cassette, another embodiment is contemplated where more than one expression cassette is used to express the desired anti-VEGF antibody sequences.

In another aspect, nucleic acid sequences which encode the antibody region amino acid sequences described herein are also provided. The coding sequences for the selected immunoglobulin domain (e.g., heavy and/or light chain(s)) may be obtained and/or synthesized or are described herein. Methods for sequencing an amino acid are known to those of skill in the art. Once the sequence of an amino acid is known, there are web-based and commercially available computer programs, as well as service based companies which back translate the amino acids sequences to nucleic acid coding sequences. See, e.g., backtranseq by EMBOSS, http://www.ebi.ac.uk/Tools/st/; Gene Infinity (http://www.geneinfinity.org/sms/sms_backtranslation.html); ExPasy (http://www.expasy.org/tools/).

In one embodiment, the RNA and/or cDNA coding sequences are designed for optimal expression in canine cells. Methods for synthesizing nucleic acids are known to those of skill in the art and may be utilized for all, or portions, of the nucleic acid constructs described herein. In one embodiment, the nucleic acid sequence encoding anti-VEGF antibody heavy chain variable region comprises SEQ ID NO: 6 or a codon optimized variant thereof. In one embodiment, the nucleic acid sequence encoding anti-VEGF antibody light chain variable region comprises SEQ ID NO: 7 or a codon optimized variant thereof. In one embodiment, the nucleic acid sequence encoding the anti-VEGF antibody heavy chain constant region comprises SEQ ID NO: 8 or a codon optimized variant thereof. In one embodiment, the nucleic acid sequence encoding the anti-VEGF antibody light chain constant region comprises SEQ ID NO: 9 or a codon optimized variant thereof.

In another embodiment, the nucleic acid sequence encoding any of the described immunoglobulin domains shares at least 50% identity with a sequence described herein (e.g., SEQ ID NO: 6, 7, 8, or 9). In another embodiment, the nucleic acid sequence encoding any of the described immunoglobulin domains shares at least 60% identity with a sequence described herein (e.g., SEQ ID NO: 6, 7, 8, or 9). In another embodiment, the nucleic acid sequence encoding any of the described immunoglobulin domains shares at least 70% identity with a sequence described herein (e.g., SEQ ID NO: 6, 7, 8, or 9). In another embodiment, the nucleic acid sequence encoding any of the described immunoglobulin domains shares at least 80% identity with a sequence described herein (e.g., SEQ ID NO: 6, 7, 8, or 9). In another embodiment, the nucleic acid sequence encoding any of the described immunoglobulin domains shares at least 90% identity with a sequence described herein (e.g., SEQ ID NO: 6, 7, 8, or 9).

In another embodiment, a nucleic acid sequence which encodes the anti-VEGF antibody heavy chain variable region of SEQ ID NO: 1 is provided. In another embodiment, a nucleic acid sequence which encodes the anti-VEGF antibody heavy chain constant region of SEQ ID NO: 2 is provided. In another embodiment, a nucleic acid sequence which encodes the anti-VEGF antibody light chain variable region of SEQ ID NO: 3 is provided. In another embodiment, a nucleic acid sequence which encodes the anti-VEGF antibody light chain constant region of SEQ ID NO: 4 is provided. It is intended that all nucleic acids encoding the described polypeptide sequences are encompassed, including nucleic acid sequences which have been optimized for expression in the desired target subject (e.g., by codon optimization).

Codon-optimized coding regions can be designed by various different methods. This optimization may be performed using methods which are available on-line (e.g., GeneArt), published methods, or a company which provides codon optimizing services, e.g., as DNA2.0 (Menlo Park, Calif.). One codon optimizing algorithm is described, e.g., in International Patent Publication No. WO 2015/012924, which is incorporated by reference herein. See also, e.g., US Patent Publication No. 2014/0032186 and US Patent Publication No. 2006/0136184. Suitably, the entire length of the open reading frame (ORF) for the product is modified. However, in some embodiments, only a fragment of the ORF may be altered (e.g., one or more of the individual immunoglobulin domains). By using one of these methods, one can apply the frequencies to any given polypeptide sequence, and produce a nucleic acid fragment of a codon-optimized coding region which encodes the polypeptide.

A number of options are available for performing the actual changes to the codons or for synthesizing the codon-optimized coding regions designed as described herein. Such modifications or synthesis can be performed using standard and routine molecular biological manipulations well known to those of ordinary skill in the art. In one approach, a series of complementary oligonucleotide pairs of 80-90 nucleotides each in length and spanning the length of the desired sequence are synthesized by standard methods. These oligonucleotide pairs are synthesized such that upon annealing, they form double stranded fragments of 80-90 base pairs, containing cohesive ends, e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides are designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they ligated together and cloned into a standard bacterial cloning vector, for example, a TOPO® vector available from Invitrogen Corporation, Carlsbad, Calif. The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final construct. The final construct is then cloned into a standard bacterial cloning vector, and sequenced. Additional methods would be immediately apparent to the skilled artisan. In addition, gene synthesis is readily available commercially.

Optionally, substitutions to the immunoglobulin domain nucleic acid or amino acid sequences may be made from the native sequences or sequences provided herein to enhance expression, targeting or for another reason. Methods and computer programs for preparing such alignments are available and well known to those of skill in the art. Substitutions may also be written as (amino acid identified by single letter code)-position #-(amino acid identified by single letter code) whereby the first amino acid is the substituted amino acid and the second amino acid is the substituting amino acid at the specified position. The terms “substitution” and “substitution of an amino acid” and “amino acid substitution” as used herein refer to a replacement of an amino acid in an amino acid sequence with another one, wherein the latter is different from the replaced amino acid. Methods for replacing an amino acid are well known to the skilled in the art and include, but are not limited to, mutations of the nucleotide sequence encoding the amino acid sequence. Methods of making amino acid substitutions in IgG are described, e.g., for WO 2013/046704, which is incorporated by reference for its discussion of amino acid modification techniques.

In another aspect, chimeric anti-VEGF antibodies and domains thereof, are provided. In one embodiment, the chimeric anti-VEGF antibody includes murine variable regions and canine constant regions. In one embodiment, the anti-VEGF antibody heavy chain immunoglobulin domain comprises a variable chain sequence and at least one constant chain sequence. In another embodiment, the anti-VEGF antibody heavy chain variable fragment immunoglobulin amino acid sequence is SEQ ID NO: 1 (CDRs underlined): E I Q L V Q S G P E L K Q P G E T V R I S C K A S G Y T F T N Y G M N W V K Q A P G K G L K W M G W I N T Y T G E P T Y A A D F K R R F T F S L E T S A S T A Y L Q I S N L K N D D T A T Y F C A K Y P H Y Y G S S H W Y F D V W G A G T T V T V S S A. In another embodiment, the anti-VEGF antibody heavy chain variable fragment immunoglobulin has a sequence sharing 80%, 85%, 90%, 95%, 96%, 97%, 98%, 99% or greater identity with SEQ ID NO: 1. In another embodiment, a nucleic acid sequence encoding the anti-VEGF antibody heavy chain immunoglobulin is provided. In another embodiment, the anti-VEGF antibody comprises the CDRs underlined above.

In another embodiment, the anti-VEGF antibody heavy chain constant fragment immunoglobulin amino acid sequence is SEQ ID NO: 2: S T T A P S V F P L A P S C G S T S G S T V A L A C L V S G Y F P E P V T V S W N S G S L T S G V H T F P S V L Q S S G L H S L S S M V T V P S S R W P S E T F T C N V V H P A S N T K V D K P V F N E C R C T D T P P C P V P E P L G G P S V L I F P P K P K D I L R I T R T P E V T C V V L D L G R E D P E V Q I S W F V D G K E V H T A K T Q S R E Q Q F N G T Y R V V S V L P I E H Q D W L T G K E F K C R V N H I D L P S P I E R T I S K A R G R A H K P S V Y V L P P S P K E L S S S D T V S I T C L I K D F Y P P D I D V E W Q S N G Q Q E P E R K H R M T P P Q L D E D G S Y F L Y S K L S V D K S R W Q Q G D P F T C A V M H E T L Q N H Y T D L S L S H S P G K. In another embodiment, the anti-VEGF antibody heavy chain canine constant region immunoglobulin has a sequence sharing 80%, 85%, 90%, 95%, 96%, 97%, 98%, 99% or greater identity with SEQ ID NO: 2. In another embodiment, a nucleic acid sequence encoding the anti-VEGF antibody canine heavy chain constant region immunoglobulin is provided.

In one embodiment, the anti-VEGF antibody light chain immunoglobulin domain comprises a variable chain sequence and at least one canine constant chain sequence. In one embodiment, the anti-VEGF antibody light chain variable fragment immunoglobulin amino acid sequence is SEQ ID NO: 3 (CDRs underlined): D I Q M T Q T T S S L S A S L G D R V I I S C S A S Q D I S N Y L N W Y Q Q K P D G T V K V L I Y F T S S L H S G V P S R F S G S G S G T D Y S L T I S N L E P E D I A T Y Y C Q Q Y S T V P W T F G G G T K L E I K R. In another embodiment, the anti-VEGF antibody light chain variable region has a sequence sharing 80%, 85%, 90%, 95%, 96%, 97%, 98%, 99% or greater identity with SEQ ID NO: 3. In another embodiment, a nucleic acid sequence encoding the anti-VEGF antibody light chain variable region is provided. In another embodiment, the anti-VEGF antibody comprises the CDRs underlined above.

In one embodiment, the anti-VEGF antibody light chain canine constant region immunoglobulin amino acid sequence is SEQ ID NO: 4: N D A Q P A V Y L F Q P S P D Q L H T G S A S V V C L L N S F Y P K D I N V K W K V D G V I Q D T G I Q E S V T E Q D K D S T Y S L S S T L T M S S T E Y L S H E L Y S C E I T H K S L P S T L I K S F Q R S E C. In another embodiment, the anti-VEGF antibody light chain constant region has a sequence sharing 80%, 85%, 90%, 95%, 96%, 97%, 98%, 99% or greater identity with SEQ ID NO: 4. In another embodiment, a nucleic acid sequence encoding the anti-VEGF antibody light chain constant region is provided.

In one embodiment, the chimeric anti-VEGF antibody comprises one or more of SEQ ID NO: 1, SEQ ID NO: 2, SEQ ID NO: 3, or SEQ ID NO: 4. In another embodiment, the chimeric anti-VEGF antibody comprises one or more sequences sharing 80%, 85%, 90%, 95%, 96%, 97%, 98%, 99% or greater identity with one or more of SEQ ID NO: 1, SEQ ID NO: 2, SEQ ID NO: 3, or SEQ ID NO: 4.

The term “amino acid substitution” and its synonyms described above are intended to encompass modification of an amino acid sequence by replacement of an amino acid with another, substituting amino acid. The substitution may be a conservative or non-conservative substitution depending on the desired outcome. The term conservative, in referring to two amino acids, is intended to mean that the amino acids share a common property recognized by one of skill in the art. The term non-conservative, in referring to two amino acids, is intended to mean that the amino acids which have differences in at least one property recognized by one of skill in the art. For example, such properties may include amino acids having hydrophobic nonacidic side chains, amino acids having hydrophobic side chains (which may be further differentiated as acidic or nonacidic), amino acids having aliphatic hydrophobic side chains, amino acids having aromatic hydrophobic side chains, amino acids with polar neutral side chains, amino acids with electrically charged side chains, amino acids with electrically charged acidic side chains, and amino acids with electrically charged basic side chains. Both naturally occurring and non-naturally occurring amino acids are known in the art and may be used as substituting amino acids in embodiments. Thus, a conservative amino acid substitution may involve changing a first amino acid having a hydrophobic side chain with a different amino acid having a hydrophobic side chain; whereas a non-conservative amino acid substitution may involve changing a first amino acid with an acidic hydrophobic side chain with a different amino acid having a different side chain, e.g., a basic hydrophobic side chain or a hydrophilic side chain. Still other conservative or non-conservative changes may be determined by one of skill in the art. In still other embodiments, the substitution at a given position will be to an amino acid, or one of a group of amino acids, that will be apparent to one of skill in the art in order to accomplish an objective identified herein. As used herein, the term “% identity” may refer to a specific number of amino acid substitutions. For example, for SEQ ID NO: 1, which has 124 amino acids, a sequence sharing “at least 90% identity” my have 1, 2, 3, 4, 5, 6, 7, 8, 9, 10, 11, or 12 amino acid substitutions as compared to the native sequence. Such definition is contemplated herein.

In order to express a selected immunoglobulin domain, a nucleic acid molecule may be designed which contains codons which have been selected for optimal expression of the immunoglobulin domain polypeptides in a selected mammalian species, e.g., canines. Further, the nucleic acid molecule may include a heterologous leader sequence for each heavy chain and light chain of the selected antibody. In one embodiment, the leader sequence encodes the IL-2 leader peptide fused upstream of the heavy chain polypeptides composed of the variable and constant regions and a second leader IL-2 leader peptide fused upstream of the light chain polypeptide composed of the variable region and constant region. In one embodiment, the first and second leader sequences are the same. In another embodiment, the first and second leader sequences are different. In one embodiment, the leader sequence is SEQ ID NO: 5: M Y R M Q L L S C I A L S L A L V T N S. However, another heterologous leader sequence may be substituted for one or both of the IL-2 signal/leader peptides. Signal/leader peptides may be the same or different for each of the heavy chain and light chain immunoglobulin domain constructs. These may be signal sequences which are natively found in an immunoglobulin (e.g., IgG), or may be from a heterologous source. Such heterologous sources may be a cytokine (e.g., IL-2, IL12, IL18, or the like), insulin, albumin, β-glucuronidase, alkaline protease or the fibronectin secretory signal peptides, or sequences from tissue specific secreted proteins, amongst others.

As used herein, an “expression cassette” refers to a nucleic acid molecule which comprises an immunoglobulin gene(s) (e.g., an immunoglobulin variable region, an immunoglobulin constant region, a full-length light chain, a full-length heavy chain or another fragment of an immunoglobulin construct), promoter, and may include other regulatory sequences therefor, which cassette may be delivered via a genetic element (e.g., a plasmid) to a packaging host cell and packaged into the capsid of a viral vector (e.g., an AAV or other parvovirus particle) or the envelope of an enveloped virus. Typically, such an expression cassette for generating a viral vector contains the immunoglobulin sequences described herein flanked by packaging signals of the viral genome and other expression control sequences. Such sequences, together, may be referred to herein, as the vector genome. However, it is intended that the terms “expression cassette” and “vector genome” may be used interchangeably. In one embodiment, the expression cassette comprises at least a first open reading frame (ORF) and optionally a second ORF. An ORF may contain one, two, three or four antibody domains. For example, the ORF may contain a full-length heavy chain. Alternatively, an ORF may contain one or two antibody domains. For example, the ORF may contain a heavy chain variable domain and a single heavy chain constant domain. In another example, the ORF may contain a heavy chain variable domain and three heavy chain constant domains. In another example, the ORF may contain a light chain variable and a light chain constant region. Thus, an expression cassette may be designed to be bicistronic, i.e., to contain regulatory sequences which direct expression of the ORFs thereon from shared regulatory sequences. In this instance, the two ORFs are typically separated by a linker. Suitable linkers, such as an internal ribozyme binding site (IRES) and/or a furin-2a self-cleaving peptide linker (F2a), [see, e.g., Radcliffe and Mitrophanous, Gene Therapy (2004), 11, 1673-1674] are known in the art. In one embodiment, the linker is an IRES. In another embodiment, the linker is an F2a. In another embodiment, each ORF is contained within a separate expression cassette.

Suitably, the ORF are operably linked to regulatory control sequences which direct expression in a target cell. Such regulatory control sequences may include a polyA, a promoter, and an enhancer. In order to facilitate co-expression from an AAV vector, at least one of the enhancer and/or polyA sequence may be shared by the first and second ORF.

In addition to a sequence encoding at least one anti-VEGF immunoglobulin domain (or the entire anti-VEGF antibody), the expression cassette includes sequences which direct expression of the VEGF domain/antibody in a host cell. Suitable regulatory control sequences may be selected and obtained from a variety of sources. In one embodiment, the vector comprises a promoter. In one embodiment, the promoter is a constitutive promoter. Examples of constitutive promoters suitable for controlling expression of the antibody domains include, but are not limited to chicken β-actin (CB) or beta actin promoters from other species, human cytomegalovirus (CMV) promoter, the early and late promoters of simian virus 40 (SV40), U6 promoter, metallothionein promoters, EFla promoter, ubiquitin promoter, hypoxanthine phosphoribosyl transferase (HPRT) promoter, dihydrofolate reductase (DHFR) promoter (Scharfmann et al., Proc. Natl. Acad. Sci. USA 88:4626-4630 (1991), adenosine deaminase promoter, phosphoglycerol kinase (PGK) promoter, pyruvate kinase promoter phosphoglycerol mutase promoter, the β-actin promoter (Lai et al., Proc. Natl. Acad. Sci. USA 86: 10006-10010 (1989), UbB, UbC, the long terminal repeats (LTR) of Moloney Leukemia Virus and other retroviruses, the thymidine kinase promoter of Herpes Simplex Virus and other constitutive promoters known to those of skill in the art. Examples of tissue- or cell-specific promoters suitable for use in the present invention include, but are not limited to, endothelin-I (ET-I) and Flt-I, which are specific for endothelial cells, FoxJ1 (that targets ciliated cells).

Although less desired, inducible promoters suitable for controlling expression of the antibody domains including promoters responsive to exogenous agents (e.g., pharmacological agents) or to physiological cues may be utilized. These response elements include, but are not limited to a hypoxia response element (HRE) that binds HIF-Iα and β, a metal-ion response element such as described by Mayo et al. (1982, Cell 29:99-108); Brinster et al. (1982, Nature 296:39-42) and Searle et al. (1985, Mol. Cell. Biol. 5:1480-1489); or a heat shock response element such as described by Nouer et al. (in: Heat Shock Response, ed. Nouer, L., CRC, Boca Raton, Fla., ppI67-220, 1991)

In one embodiment, expression of an open reading frame is controlled by a regulatable promoter that provides tight control over the transcription of the ORF (gene), e.g., a pharmacological agent, or transcription factors activated by a pharmacological agent or in alternative embodiments, physiological cues. Examples of regulatable promoters which are ligand-dependent transcription factor complexes that may be used include, without limitation, members of the nuclear receptor superfamily activated by their respective ligands (e.g., glucocorticoid, estrogen, progestin, retinoid, ecdysone, and analogs and mimetics thereof) and rTTA activated by tetracycline. Examples of such systems, include, without limitation, the ARGENT™ Transcriptional Technology (ARIAD Pharmaceuticals, Cambridge, Mass.). Examples of such promoter systems are described, e.g., in WO 2012/145572, which is incorporated by reference herein. In other embodiments, small RNA based switches are described in http://www/ncbi.nlm.nih.gov/pubmed/25605380.

Still other promoters may include, e.g., human cytomegalovirus (CMV) immediate-early enhancer/promoter, the SV40 early enhancer/promoter, the JC polymovirus promoter, myelin basic protein (MBP) or glial fibrillary acidic protein (GFAP) promoters, herpes simplex virus (HSV-1) latency associated promoter (LAP), rouse sarcoma virus (RSV) long terminal repeat (LTR) promoter, neuron-specific promoter (NSE), platelet derived growth factor (PDGF) promoter, hSYN, melanin-concentrating hormone (MCH) promoter, CBA, glial fibriallary acidic protein (GFAP) promoter, matrix metalloprotein promoter (MPP), and the chicken beta-actin promoter. The promoters may the same or different for each expression cassette.

In one embodiment, expression of each the one or more ORFs is controlled by the same promoter (e.g., when used with a linker such as an IRES). In another embodiment, the expression of each ORF is controlled by a separate promoter. Each promoter may be separately selected based on the description herein.

In one embodiment, a minimal promoter and/or a minimal polyA may be utilized to conserve size. As used herein, the term “minimal promoter” means a short DNA sequence comprised of a TATA-box and other sequences that serve to specify the site of transcription initiation, to which regulatory elements are added for control of expression. In one embodiment, a promoter refers to a nucleotide sequence that includes a minimal promoter plus regulatory elements that are capable of controlling the expression of a coding sequence or functional RNA. This type of promoter sequence consists of proximal and more distal upstream elements, the latter elements often referred to as enhancers. In one embodiment, the minimal promoter is a Cytomegalovirus (CMV) minimal promoter. In another embodiment, the minimal promoter is derived from human CMV (hCMV) such as the hCMV immediate early promoter derived minimal promoter (see, US 20140127749, and Gossen and Bujard (Proc. Natl. Acad. Sci. USA, 1992, 89: 5547-5551), which are incorporated herein by reference). In another embodiment, the minimal promoter is derived from a viral source such as, for example: SV40 early or late promoters, cytomegalovirus (CMV) immediate early promoters, or Rous Sarcoma Virus (RSV) early promoters; or from eukaryotic cell promoters, for example, beta actin promoter (Ng, Nuc. Acid Res. 17:601-615, 1989; Quitsche et al., J. Biol. Chem. 264:9539-9545, 1989), GADPH promoter (Alexander, M. C. et al., Proc. Nat. Acad. Sci. USA 85:5092-5096, 1988, Ercolani, L. et al., J. Biol. Chem. 263:15335-15341, 1988), TK-1 (thymidine kinase) promoter, HSP (heat shock protein) promoters, UbB or UbC promoter, PGK, Ef1-alpha promoter or any eukaryotic promoter containing a TATA box (US Published Application No. 2014/0094392). In another embodiment, the minimal promoter includes a mini-promoter, such as the CLDNS mini-promoter described in US Published Application No. 2014/0065666. In another embodiment, the minimal promoter is the Thymidine Kinase (TK) promoter. In one embodiment, the minimal promoter is tissue specific, such as one of the muscle-cell specific promoters minimal TnISlow promoter, a minimal TnlFast promoter or a muscle creatine kinase promoter (US Published Application No. 2012/0282695). Each of these documents is incorporated herein by reference.

In one embodiment, a polyadenylation signal is included. In one embodiment, a wild-type or synthetic polyA may be selected. In another embodiment, the polyadenylation (poly(A)) signal is a minimal poly(A) signal, i.e., the minimum sequence required for efficient polyadenylation. In one embodiment, the minimal poly(A) is a synthetic poly(A), such as that described in Levitt et al, Genes Dev., 1989 Jul., 3(7):1019-25; and Xia et al, Nat Biotechnol. 2002 October; 20(10):1006-10. Epub 2002 Sep. 16. In another embodiment, the poly(A) is derived from the rabbit beta-globin poly(A). In one embodiment, the polyA acts bidirectionally (An et al, 2006, PNAS, 103(49): 18662-18667. In one embodiment, the poly(A) is derived from the SV40 early poly A signal sequence. In one embodiment, the poly(A) is derived from the SV40 late poly A signal sequence. Each of these documents is incorporated herein by reference.

Optionally, a single enhancer, or the same enhancer, may regulate the transcription of multiple heterologous genes (i.e., the heavy chain immunoglobulin and the light chain immunoglobulin) in the plasmid construct. Various enhancers suitable for use in the invention are known in the art and include, for example, the CMV early enhancer, Hoxc8 enhancer, nPE1 and nPE2. Additional enhancers useful herein are described in Andersson et al, Nature, 2014 March, 507(7493):455-61, which is incorporated herein by reference. Still other enhancer elements may include, e.g., an apolipoprotein enhancer, a zebrafish enhancer, a GFAP enhancer element, and tissue specific enhancers such as described in WO 2013/1555222, woodchuck post hepatitis post-transcriptional regulatory element. Additionally, or alternatively, other, e.g., the hybrid human cytomegalovirus (HCMV)-immediate early (IE)-PDGR promoter or other promoter—enhancer elements may be selected. To enhance expression the other elements can be introns (like promega intron or chimeric chicken globin-human immunoglobulin intron). Other enhancers useful herein can be found in the Mammalian Promoter/Enhancer Database found at http://promoter.cdb.riken.jp/.

The constructs described herein may further contain other expression control or regulatory sequences such as, e.g., appropriate transcription initiation, termination, promoter and enhancer sequences; introns; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product.

These control sequences are “operably linked” to the immunoglobulin construct gene sequences. As used herein, the term “operably linked” refers to both expression control sequences that are contiguous with the gene of interest and expression control sequences that act in trans or at a distance to control the gene of interest.

In one embodiment, each promoter is located either adjacent (either to the left or the right (or 5′ or 3′)) to the enhancer sequence and the polyA sequences are located adjacent to the ITRs, with the ORFs there between. While, in one embodiment, the heavy chain sequences are expressed first, the order of the ORFs may be varied, as may the immunoglobulin domains encoded thereby. For example, the light chain constant and variable sequences may be located to the left of the linker and the heavy chain may be encoded by ORFs located to the right of the linker. Alternatively, the heavy chain may be located to the left of the linker and the ORFs to the right of the linker may encode a light chain. Alternatively, the opposite configuration is possible. In one embodiment, the expression cassette contains sequences encoding a promoter followed by sequences encoding the heavy chain, followed by a F2A sequence, followed by sequences encoding the light chain. Illustrative examples of this configuration can be found in the plasmid sequence set forth in SEQ ID NO: 11 and SEQ ID NO: 12. In another embodiment, the expression cassette contains sequences encoding a promoter followed by sequences encoding the light chain, followed by an IRES sequence, followed by sequences encoding the heavy chain. An Illustrative example of this configuration can be found in the plasmid sequence set forth in SEQ ID NO: 13. In another embodiment, the rAAV has packaged within the selected AAV capsid, a nucleic acid molecule comprising: a 5′ AAV inverted terminal repeat sequence (ITR), a promoter, a signal peptide operably linked to a murine VEGF immunoglobulin variable heavy chain and a canine VEGF constant heavy chain, an IRES, a signal peptide operably linked to a murine VEGF variable light chain, and a 3′ AAV ITR. In one embodiment, the AAV capsid is AAV8. In a further embodiment, the ITRs are from AAV2, or a different source which is different from the AAV capsid source.

In one embodiment, the vector is an adeno-associated virus vector. A recombinant AAV vector (AAV viral particle) may comprise, packaged within an AAV capsid, a nucleic acid molecule expressing a functional antibody as described in this specification. An expression cassette may contain regulatory elements for an open reading frame(s) within each expression cassette and the nucleic acid molecule may optionally contain additional regulatory elements.

The AAV vector may contain a full-length AAV 5′ inverted terminal repeat (ITR) and a full-length 3′ ITR. A shortened version of the 5′ ITR, termed ΔITR, has been described in which the D-sequence and terminal resolution site (trs) are deleted. The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety. In another embodiment, a self-complementary AAV is used.

Where a pseudotyped AAV is to be produced, the ITRs are selected from a source which differs from the AAV source of the capsid. For example, AAV2 ITRs may be selected for use with an AAV capsid having a particular efficiency for a selected cellular receptor, target tissue or viral target. In one embodiment, the ITR sequences from AAV2, or the deleted version thereof (ΔITR), are used for convenience and to accelerate regulatory approval. However, ITRs from other AAV sources may be selected. Where the source of the ITRs is from AAV2 and the AAV capsid is from another AAV source, the resulting vector may be termed pseudotyped. However, other sources of AAV ITRs may be utilized.

A variety of AAV capsids have been described. Methods of generating AAV vectors have been described extensively in the literature and patent documents, including, e.g., WO 2003/042397; WO 2005/033321, WO 2006/110689; U.S. Pat. No. 7,588,772 B2. The source of AAV capsids may be selected from an AAV which targets a desired tissue. For example, suitable AAV may include, e.g., AAV9 [U.S. Pat. No. 7,906,111; US 2011-0236353-A1], rh10 [WO 2003/042397] and/or hu37 [see, e.g., U.S. Pat. No. 7,906,111; US 2011-0236353-A1]. However, other AAV, including, e.g., AAV1, AAV2, AAV3, AAV4, AAVS, AAV6, AAV7, AAV8, [U.S. Pat. Nos. 7,790,449; 7,282,199] and others, including variants thereof. In one embodiment, the AAV capsid is an AAV8 capsid or variant thereof. In one embodiment, when referring to an AAV capsid, the term “variant” refers to capsids sharing at least about 90% identity, 95% identity, 97% identity, 98% identity, 99% identity or greater with the named AAV capsid. For example, in one embodiment, the AAV capsid is the AAV8 capsid identified by NCBI Reference Sequence: YP_077180.1 or a sequence sharing at least 95% identity therewith. However, other sources of AAV capsids and other viral elements may be selected, as may other immunoglobulin constructs and other vector elements.

A single-stranded AAV viral vector is provided. Methods for generating and isolating AAV viral vectors suitable for delivery to a subject are known in the art. See, e.g., U.S. Pat. Nos. 7,790,449; 7,282,199; WO 2003/042397; WO 2005/033321, WO 2006/110689; and U.S. Pat. No. 7,588,772 B2]. In one system, a producer cell line is transiently transfected with a construct that encodes the transgene flanked by ITRs and a construct(s) that encodes rep and cap. In a second system, a packaging cell line that stably supplies rep and cap is transiently transfected with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

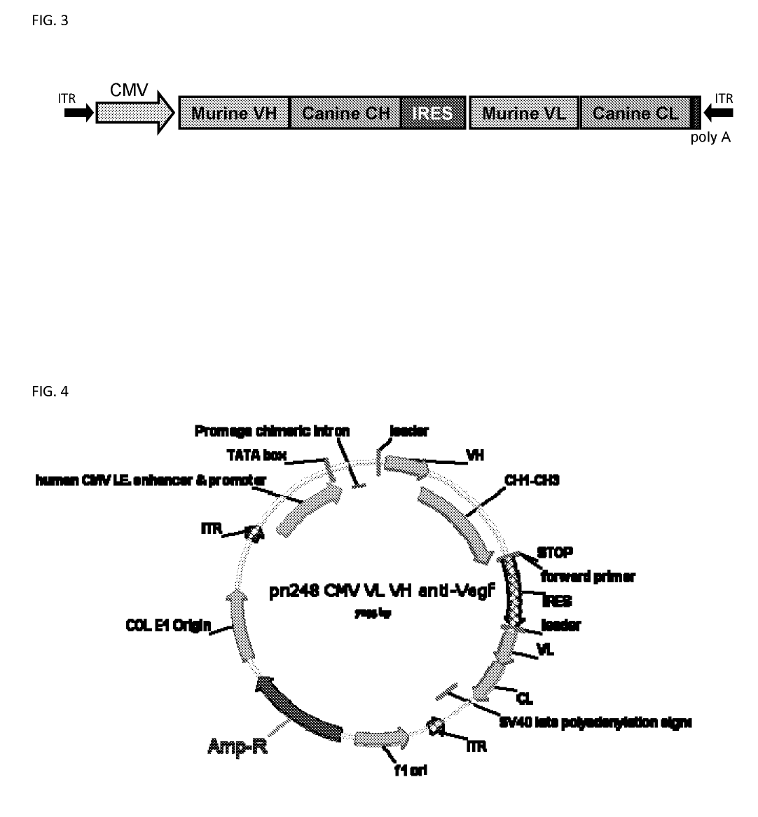

For use in producing an AAV viral vector (e.g., a recombinant (r) AAV)), the expression cassettes can be carried on any suitable vector, e.g., a plasmid, which is delivered to a packaging host cell. The plasmids useful in this invention may be engineered such that they are suitable for replication and packaging in prokaryotic cells, mammalian cells, or both. Suitable transfection techniques and packaging host cells are known and/or can be readily designed by one of skill in the art. An example of a plasmid used to produce the viral vector utilized in the examples is shown in FIG. 3 and in SEQ ID NO: 10.

Methods for generating and isolating AAVs suitable for use as vectors are known in the art. See generally, e.g., Grieger & Samulski, 2005, “Adeno-associated virus as a gene therapy vector: Vector development, production and clinical applications,” Adv. Biochem. Engin/Biotechnol. 99: 119-145; Buning et al., 2008, “Recent developments in adeno-associated virus vector technology,” J. Gene Med. 10:717-733; and the references cited below, each of which is incorporated herein by reference in its entirety. For packaging a transgene into virions, the ITRs are the only AAV components required in cis in the same construct as the nucleic acid molecule containing the expression cassettes. The cap and rep genes can be supplied in trans.

As described above, the term “about” when used to modify a numerical value means a variation of ±10%, unless otherwise specified.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 90%, 91%, 92%, 93%, 94%, 95%, 96%, 97%, 98%, 99%, or higher identity over a specified region (e.g., any one of the ORFs provided herein when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). As another example, polynucleotide sequences can be compared using Fasta, a program in GCG Version 6.1. Fasta provides alignments and percent sequence identity of the regions of the best overlap between the query and search sequences. For instance, percent sequence identity between nucleic acid sequences can be determined using Fasta with its default parameters (a word size of 6 and the NOPAM factor for the scoring matrix) as provided in GCG Version 6.1, herein incorporated by reference. Generally, these programs are used at default settings, although one skilled in the art can alter these settings as needed. Alternatively, one of skill in the art can utilize another algorithm or computer program that provides at least the level of identity or alignment as that provided by the referenced algorithms and programs. This definition also refers to, or can be applied to, the compliment of a sequence. The definition also includes sequences that have deletions and/or additions, as well as those that have substitutions. As described below, the preferred algorithms can account for gaps and the like. Preferably, identity exists over a region that is at least about 25, 50, 75, 100, 150, 200 amino acids or nucleotides in length, and oftentimes over a region that is 225, 250, 300, 350, 400, 450, 500 amino acids or nucleotides in length or over the full-length of an amino acid or nucleic acid sequences.

Typically, when an alignment is prepared based upon an amino acid sequence, the alignment contains insertions and deletions which are so identified with respect to a reference AAV sequence and the numbering of the amino acid residues is based upon a reference scale provided for the alignment. However, any given AAV sequence may have fewer amino acid residues than the reference scale. In the present invention, when discussing the parental sequence, the term “the same position” or the “corresponding position” refers to the amino acid located at the same residue number in each of the sequences, with respect to the reference scale for the aligned sequences. However, when taken out of the alignment, each of the proteins may have these amino acids located at different residue numbers. Alignments are performed using any of a variety of publicly or commercially available Multiple Sequence Alignment Programs. Sequence alignment programs are available for amino acid sequences, e.g., the “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

In one embodiment, the expression cassettes described herein are engineered into a genetic element (e.g., a shuttle plasmid) which transfers the immunoglobulin construct sequences carried thereon into a packaging host cell for production a viral vector. In one embodiment, the selected genetic element may be delivered to a an AAV packaging cell by any suitable method, including transfection, electroporation, liposome delivery, membrane fusion techniques, high velocity DNA-coated pellets, viral infection and protoplast fusion. Stable AAV packaging cells can also be made. Alternatively, the expression cassettes may be used to generate a viral vector other than AAV, or for production of mixtures of antibodies in vitro. The methods used to make such constructs are known to those with skill in nucleic acid manipulation and include genetic engineering, recombinant engineering, and synthetic techniques. See, e.g., Molecular Cloning: A Laboratory Manual, ed. Green and Sambrook, Cold Spring Harbor Press, Cold Spring Harbor, N.Y. (2012).

As used herein a vector may be any suitable genetic element which transfects, transduces or infects a host cell and expresses the immunoglobulins which assemble into a functional antibody. Such vectors may be selected from a lentiviral vector, a baculovirus vector, a parvovirus vector, a plasmid, modified RNA, and a DNA molecule where mRNA and DNA may be in a form of nanoparticles.

The vector is preferably suspended in a physiologically compatible carrier, for administration to a human or non-human mammalian patient. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, maltose, and water. Optionally, the compositions of the invention may contain, in addition to the rAAV and carrier(s), other conventional pharmaceutical ingredients, such as preservatives, or chemical stabilizers.

Also provided are compositions which include the viral vector constructs described herein. The pharmaceutical compositions described herein are designed for delivery to subjects in need thereof by any suitable route or a combination of different routes. Any suitable method or route can be used to administer an AAV-containing composition as described herein, and optionally, to co-administer other active drugs or therapies in conjunction with the AAV-mediated antibodies described herein. Routes of administration include, for example, systemic, oral, inhalation, intranasal, intratracheal, intraarterial, intraocular, intravenous, intramuscular, subcutaneous, intradermal, and other parental routes of administration. The viral vectors described herein may be delivered in a single composition or multiple compositions. Optionally, two or more different AAV may be delivered, or multiple viruses [see, e.g., WO 2011/126808 and WO 2013/049493]. In another embodiment, multiple viruses may contain different replication-defective viruses (e.g., AAV and adenovirus).

In the case of AAV viral vectors, quantification of the genome copies (“GC”) may be used as the measure of the dose contained in the formulation. Any method known in the art can be used to determine the genome copy (GC) number of the replication-defective virus compositions of the invention. One method for performing AAV GC number titration is as follows: Purified AAV vector samples are first treated with DNase to eliminate un-encapsidated AAV genome DNA or contaminating plasmid DNA from the production process. The DNase resistant particles are then subjected to heat treatment to release the genome from the capsid. The released genomes are then quantitated by real-time PCR using primer/probe sets targeting specific region of the viral genome (usually poly A signal). Other suitable methods include digital PCT, digital droplet PCR, and optimized qPCR.

Also, the replication-defective virus compositions can be formulated in dosage units to contain an amount of replication-defective virus that is in the range of about 1.0×10⁹ GC to about 1.0×10¹⁵ GC. In another embodiment, this amount of viral genome may be delivered in split doses. In one embodiment, the dosage is about 1.0×10¹⁰ GC to about 1.0×10¹² GC for an average small canine subject of about 5 kg. In one embodiment, the dosage is about 1.0×10¹¹ GC to about 5.0×10¹³ GC for an average medium canine subject of about 20 kg. In one embodiment, the dosage is about 1.0×10¹² GC to about 1.0×10¹⁴ GC for an average large canine subject of about 50 kg. The average canine ranges from about 5 to about 50 kg in body weight. In one embodiment, the dosage is about 1×10¹² GC/kg. In one embodiment, the dosage is about 1.0×10¹¹ GC to 1.0×10¹⁴ GC for a subject. In another embodiment, the dose about 1×10¹³ GC. For example, the dose of AAV virus may be about 1×10¹² GC, about 5×10¹² GC, about 1×10¹³ GC, about 5×10¹³ GC, or about 1×10¹⁴ GC. In another example, the constructs may be delivered in an amount of about 0.001 mg to about 10 mg per mL. In one embodiment, the constructs may be delivered in volumes from 1 μL to about 100 mL for a veterinary subject. See, e.g., Diehl et al, J. Applied Toxicology, 21:15-23 (2001) for a discussion of good practices for administration of substances to various veterinary animals. This document is incorporated herein by reference. As used herein, the term “dosage” can refer to the total dosage delivered to the subject in the course of treatment, or the amount delivered in a single (of multiple) administration.

The above-described recombinant vectors may be delivered to host cells according to published methods. The rAAV, preferably suspended in a physiologically compatible carrier, carrier, diluent, excipient and/or adjuvant, may be administered to a desired subject including without limitation, a cat, dog, or other non-human mammalian subject. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present invention.

Optionally, the compositions of the invention may contain, in addition to the rAAV and/or variants and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

In one aspect, the vectors provided herein express effective levels of functional antibody when delivered in a dose of about 3 mL or less, 1 mL or less, 0.5 mL, or less, e.g., in the range of about 100 μL to 250 μL. Thus, the vectors provided herein are highly efficient at providing therapeutic levels of antibody at doses which are convenient for metered doses, or in products or kits containing pre-measured doses.

The viral vectors and other constructs described herein may be used in preparing a medicament for delivering a chimeric anti-VEGF antibody construct to a subject in need thereof, and/or for treating hemangiosarcoma in a subject. Thus, in another aspect a method of treating hemangiosarcoma is provided. The method includes administering a composition as described herein to a subject in need thereof. In one embodiment, the composition includes a viral vector containing an anti-VEGF antibody expression cassette, as described herein. In one embodiment, the subject is a mammal. In another embodiment, the subject is a canine.

In another embodiment, a method for treating hemangiosarcoma in a canine is provided. The method includes administering a viral vector comprising a nucleic acid molecule comprising a sequence encoding a chimeric anti-VEGF antibody to the subject.

In another embodiment, a method for treating cancer in a subject is provided. The method includes administering a viral vector comprising a nucleic acid molecule comprising a sequence encoding an anti-VEGF antibody to the subject. In one embodiment, the cancer is a cancer in which VEGF is implicated, e.g., upregulated. For example, it is believed that VEGF is implicated in angiogenesis, vascular permeability and tumorigenesis. See, e.g., Goel and Mercurio, VEGF targets the tumor cell, Nature Reviews Cancer, 13:871-82 (2013) which is incorporated herein by reference. In another embodiment, the cancer is a cancer in which abnormal levels of vascularization driven by VEGF are demonstrated. In various embodiments of the methods and compositions described herein, the cancer can include, without limitation, breast cancer, lung cancer, prostate cancer, colorectal cancer, brain cancer, esophageal cancer, stomach cancer, bladder cancer, pancreatic cancer, cervical cancer, head and neck cancer, ovarian cancer, melanoma, acute and chronic lymphocytic and myelocytic leukemia, myeloma, Hodgkin's and non-Hodgkin's lymphoma, and multidrug resistant cancer. In one embodiment, the cancer is a drug resistant cancer. In one embodiment, the subject is a canine.

In another embodiment, a method for treating macular degeneration in a subject is provided. The method includes administering a viral vector comprising a nucleic acid molecule comprising a sequence encoding an anti-VEGF antibody to the subject. In one embodiment, the subject is a canine. In one embodiment, the macular degeneration is an X-linked macular degeneration. In another embodiment, the macular degeneration is age related macular degeneration.

A course of treatment may optionally involve repeat administration of the same viral vector (e.g., an AAV8 vector) or a different viral vector (e.g., an AAV8 and an AAVrh10) expressing an anti-VEGF antibody as described herein. Still other combinations may be selected using the viral vectors described herein. Optionally, the composition described herein may be combined in a regimen involving one or more of the following: cancer drugs (including e.g., doxorubicin, vincristine+doxorubicin+cyclophosphamide (VAC), or other chemotherapeutic drugs), surgery to remove or reduce the tumor, radiation, medications including carprofen, deracoxib, doxycycline (see, e.g., Hammer A S, Couto C G, Filppi J, et. al. Efficacy and toxicity of VAC chemotherapy (vincristine, doxorubicin, and cyclophosphamide) in dogs with hemangiosarcoma. J Vet Intern Med; 1991; 5:160-166; Sorenmo K U, Jeglum K A, Helfand S C. Chemotherapy of canine hemangiosarcoma with doxorubicin and cyclophosphamide. J Vet Intern Med 1993; 7:370-376; and Ogilvie G K, Powers B E, Mallinckrodt C H, et. al. Surgery and doxorubicin in dogs with hemangiosarcoma. J Vet Intern Med 1996; 10:379-384., which are incorporated herein by reference in their entirety) and polysaccharopeptide (PSP). Optionally, the compositions described herein may be combined in a regimen involving lifestyle changes including dietary and exercise regimens.

It is to be noted that the term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

As used herein, the term “about” means a variability of 10% from the reference given, unless otherwise specified.

The term “regulation” or variations thereof as used herein refers to the ability of a composition to inhibit one or more components of a biological pathway.

A “subject” is a mammal, e.g., a human, mouse, rat, guinea pig, dog, cat, horse, cow, pig, or non-human primate, such as a monkey, chimpanzee, baboon or gorilla. In one embodiment, the subject is a dog. As used herein, the term “subject” is used interchangeably with “patient”.

As used herein, “disease”, “disorder” and “condition” are used interchangeably, to indicate an abnormal state in a subject.

Unless defined otherwise in this specification, technical and scientific terms used herein have the same meaning as commonly understood by one of ordinary skill in the art and by reference to published texts, which provide one skilled in the art with a general guide to many of the terms used in the present application.

The following examples are illustrative only and are not intended to limit the present invention.

### Example 1—Construction of VEGF Vectors

The amino acid sequences of canine IgG subclass A and kappa light chain were obtained from Genbank. The amino acid sequence of the variable regions of murine antibody A.4.6.1 were combined with canine IgGA/kappa constant regions to produce an amino acid sequence of a chimeric canine antibody targeting VEGF. The amino acid sequences were backtranslated and codon optimized, followed by addition of a kozak consensus sequence, stop codon, and cloning sites. The sequences were produced by GeneArt, and cloned into a variety of expression vectors containing a CMV or CB promoter, with expression of both heavy and light chains accomplished through inclusion of a 2A peptide sequence and furin cleavage site between the polypeptide chains, or by expressing the 3′ polypeptide using an EMCV internal ribosomal entry site. The expression constructs were flanked by AAV2 ITRs. An example of a CMV promoter-containing expression vector is shown in FIG. 3 and SEQ ID NO: 10. The constructs were packaged in an AAV serotype 8 capsid by triple transfection and iodixanol gradient purification and titered by Taqman quantitative PCR.

### Example 2—In Vitro Expression and Antigen Binding

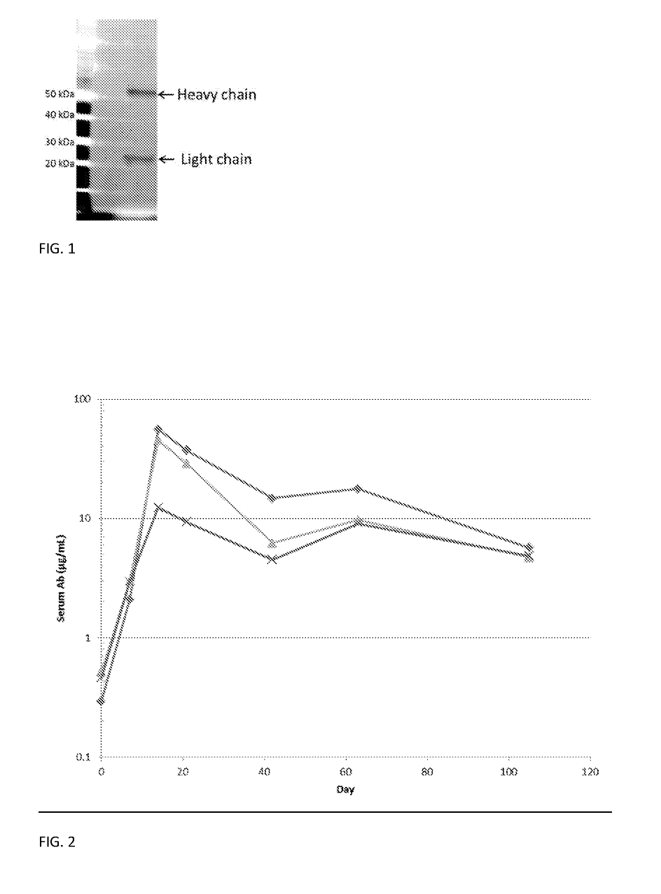

A construct containing the CMV promoter followed by the antibody heavy chain, EMCV IRES, and antibody light chain was evaluated in vitro by transient transfection of HEK 293 cells using lipofectamine 2000. The expressed antibody was purified from supernatant using a protein G column (GE) according to the manufacturer's instructions. The purified antibody was characterized by reducing SDS-PAGE with Sypro ruby staining (FIG. 1). The antibody was evaluated for binding to canine VEGF by ELISA, where the target antigen was recombinant canine VEGF (Kingfisher Bio), and bound antibody was detected by HRP-conjugated goat anti-dog secondary antibody (Peirce). Both the purified antibody and transfection supernatant showed binding to canine VEGF by ELISA.

### Example 3—AAV-Mediated Expression of a Chimeric Canine Anti-VEGF Antibody in Dogs

Three normal dogs were treated with a single intramuscular injection of 10¹² genome copies per kilogram body weight (GC/kg) AAV8 expressing the chimeric antibody. Serum was collected at indicated times and evaluated for antibody expression by VEGF-binding ELISA, with the purified antibody serving as a standard for quantification (FIG. 2).

All publications cited in this specification, as well as U.S. Provisional Patent Application No. 62/212,170, filed Aug. 31, 2015, are incorporated herein by reference. Similarly, the SEQ ID NOs which are referenced herein and which appear in the appended Sequence Listing are incorporated by reference. While the invention has been described with reference to particular embodiments, it will be appreciated that modifications can be made without departing from the spirit of the invention. Such modifications are intended to fall within the scope of the appended claims.

1: A viral vector comprising at least one nucleic acid expression cassette comprising a nucleic acid sequence encoding a functional anti-VEGF antibody which binds canine VEGF which comprises an anti-VEGF antibody heavy chain immunoglobulin of SEQ ID NO: 15 and/or an anti-VEGF antibody light chain immunoglobulin of SEQ ID NO: 14, and expression control sequences which direct expression of the immunoglobulin chains in a host cell under conditions which permit the chains to assemble into the functional antibody.

2: A viral vector comprising at least one nucleic acid expression cassette comprising a nucleic acid sequence encoding: a promoter, a first signal peptide operably linked to an anti-VEGF antibody heavy chain immunoglobulin, a linker sequence, and a second signal peptide operably linked to an anti-VEGF light chain immunoglobulin, wherein said expression cassette co-expresses the immunoglobulin chains in a host cell under conditions which permit the chains to assemble into a functional chimeric anti-VEGF antibody comprising SEQ ID NO: 14 and/or SEQ ID NO: 15.

3: The viral vector of claim 1, wherein the viral vector is an adeno-associated viral vector.

4: The viral vector of claim 3, wherein the vector has an AAV8 capsid.

5: The viral vector of claim 2, wherein said linker sequence comprises one or more of an IRES sequence, a 2A peptide sequence and a furin site.

6: The viral vector according to claim 1 wherein the chimeric anti-VEGF antibody is selected from a monoclonal antibody, an Fv, Fab, F(ab)2, F(ab)3, Fab′, Fab′-SH, F(ab′)2, an immunoadhesin, or a single chain variable fragment antibody.

7: The viral vector of claim 5, wherein the antibody is a chimeric monoclonal antibody.

8: The viral vector according to claim 1, wherein the nucleic acid sequence encoding anti-VEGF antibody heavy chain variable region comprises SEQ ID NO: 6 or a codon optimized variant thereof.

9: The viral vector according to claim 1, wherein the nucleic acid sequence encoding anti-VEGF antibody heavy chain constant region comprises SEQ ID NO: 8 or a codon optimized variant thereof.

10: The viral vector according to claim 1, wherein the nucleic acid sequence encoding anti-VEGF antibody light chain variable region comprises SEQ ID NO: 7 or a codon optimized variant thereof.

11: The viral vector according to claim 1, wherein the nucleic acid sequence encoding anti-VEGF antibody light chain constant region comprises SEQ ID NO: 9 or a codon optimized variant thereof.

12: The viral vector according to claim 1, wherein the promoter is a CMV promoter.

13: The viral vector of claim 1, wherein the expression control sequences comprise a constitutive promoter.

14: The viral vector according to claim 1, further comprising one or more of an intron, a Kozak sequence, a polyA, and a post-transcriptional regulatory elements.

15: The viral vector according to claim 1, wherein AAV capsid is selected from AAV8, rh64R1, AAV9, AAVhu.37, or rh10 and variants thereof.

16: A pharmaceutical composition comprising a pharmaceutically acceptable carrier and an rAAV according to claim 1.

17: A method for treating cancer, said method comprising administering the composition of claim 16 to a subject in need thereof.

18: The method according to claim 17, wherein said composition is administered at a dosage of about 1×10¹² GC/kg.

19: A chimeric anti-VEGF antibody comprising SEQ ID NO: 14 and SEQ ID NO: 15.

20: The chimeric antibody of claim 19, wherein the nucleic acid sequence encoding said antibody comprises at least one of SEQ ID NO: 6, SEQ ID NO: 7, SEQ ID NO: 8 or SEQ ID NO: 9, or a codon optimized variant thereof.

21-22. (canceled)


SEQUENCE LISTING (13 sequences)

SEQ ID NO: 1 - protein, 124 aa, Canis familiaris
EIQLVQSGPELKQPGETVRISCKASGYTFTNYGMNWVKQAPGKGLKWMGWINTYTGEPTYAADFKRRFTFSLETSASTAYLQISNLKNDDTATYFCAKYPHYYGSSHWYFDVWGAGTTVTVSSA

SEQ ID NO: 2 - protein, 330 aa, Mus musculus
STTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 3 - protein, 91 aa, Canis familiaris
DIQMTQTTSSLSASLGDRVIISCSASQDISNYLNWYQQKPDGTVKVLIYFTSSLHSGVPSRFSGSGSGTDYSLTISNLEPEDIATYYCQQY

SEQ ID NO: 4 - protein, 122 aa, Mus musculus
STVPWTFGGGTKLEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSEC

SEQ ID NO: 5 - protein, 20 aa, Artificial Sequence
  IL-2 leader
MYRMQLLSCIALSLALVTNS

SEQ ID NO: 6 - DNA, 372 bp, Canis familiaris
gagatccagctggtgcagtctggccccgagctgaagcagcctggcgagacagtgcggatcagctgcaaggccagcggctacaccttcaccaactacggcatgaactgggtcaagcaggcccctggcaagggcctgaagtggatgggctggatcaacacctacaccggcgagcctacctacgccgccgacttcaagcggcggttcaccttcagcctggaaaccagcgccagcaccgcctacctgcagatcagcaacctgaagaacgacgacaccgccacctacttttgcgccaagtacccccactactacggcagcagccactggtacttcgacgtgtggggagccggcaccaccgtgacagtgtcatctgcg

SEQ ID NO: 7 - DNA, 273 bp, Canis familiaris
gacatccagatgacccagaccaccagcagcctgagcgccagcctgggcgacagagtgatcatcagctgtagcgcctcccaggacatcagcaactacctgaactggtatcagcagaaacccgacggcaccgtgaaggtgctgatctacttcaccagctccctgcacagcggcgtgcccagcagattttctggcagcggctccggcaccgactacagcctgaccatctccaacctggaacccgaggatatcgccacctactactgccagcagtac

SEQ ID NO: 8 - DNA, 639 bp, Mus musculus
gacatccagatgacccagaccaccagcagcctgagcgccagcctgggcgacagagtgatcatcagctgtagcgcctcccaggacatcagcaactacctgaactggtatcagcagaaacccgacggcaccgtgaaggtgctgatctacttcaccagctccctgcacagcggcgtgcccagcagattttctggcagcggctccggcaccgactacagcctgaccatctccaacctggaacccgaggatatcgccacctactactgccagcagtacagcaccgtgccctggacctttggcggaggcaccaagctggaaatcaagcggaatgatgctcagcctgctgtgtacctttttcaaccaagccctgaccaactgcataccggcagtgcctctgtggtctgcctgcttaatagcttctatcccaaggacattaatgtgaagtggaaggttgacggcgtgatacaggataccggaattcaggaaagtgtgacagaacaagataaggatagcacctatagcctgtctagcaccctcaccatgagcagcacagagtacttgagtcatgagctgtatagctgtgagattacccacaagagtctgccaagcacccttataaaaagtttccagcgatctgagtgt

SEQ ID NO: 9 - DNA, 366 bp, Mus musculus
agcaccgtgccctggacctttggcggaggcaccaagctggaaatcaagcggaatgatgctcagcctgctgtgtacctttttcaaccaagccctgaccaactgcataccggcagtgcctctgtggtctgcctgcttaatagcttctatcccaaggacattaatgtgaagtggaaggttgacggcgtgatacaggataccggaattcaggaaagtgtgacagaacaagataaggatagcacctatagcctgtctagcaccctcaccatgagcagcacagagtacttgagtcatgagctgtatagctgtgagattacccacaagagtctgccaagcacccttataaaaagtttccagcgatctgagtgt

SEQ ID NO: 10 - DNA, 7293 bp, Artificial Sequence
  constructed sequence
  misc_feature (12)..(71)
  misc_feature (72)..(443)
  misc_feature (444)..(1433)
  enhancer (1445)..(2032): IRES
  misc_feature (2033)..(2092)
  misc_feature (2093)..(2365)
  misc_feature (2366)..(2731)
  polyA_signal (2754)..(2985): SV40\late\polyadenylation\signal
  promoter (2754)..(2985): human\CMV\I.E.\enhancer\&\promoter
  misc_feature (3050)..(3179): 3′ ITR (complement)
  misc_feature (6001)..(6130): 5′ ITR
  TATA_signal (6897)..(6901)
  Intron (7047)..(7179): Promega\chimeric\intron
ctagacccaccatgtacaggatgcaactcctgtcttgcattgcactaagtcttgcacttgtcacaaacagtgagatccagctggtgcagtctggccccgagctgaagcagcctggcgagacagtgcggatcagctgcaaggccagcggctacaccttcaccaactacggcatgaactgggtcaagcaggcccctggcaagggcctgaagtggatgggctggatcaacacctacaccggcgagcctacctacgccgccgacttcaagcggcggttcaccttcagcctggaaaccagcgccagcaccgcctacctgcagatcagcaacctgaagaacgacgacaccgccacctacttttgcgccaagtacccccactactacggcagcagccactggtacttcgacgtgtggggagccggcaccaccgtgacagtgtcatctgcgtcgaccacagccccctctgtgttccccctggccccttcctgtgggtcaacctctggcagcacagtggccctggcgtgtcttgtgtctggctacttccctgaacctgtgacagtcagctggaacagcggaagcctgacctctggagtgcacaccttccccagtgtcctgcaaagctcaggcctgcacagcctgtcaagtatggtgacagtgcccagtagcaggtggccttctgaaacctttacctgcaacgtggtgcaccctgcatccaacaccaaagtggataagcctgttttcaatgagtgcagatgcacagatacacctccctgccctgtgcctgagcctctgggaggaccatcagtcctgatcttccctccaaagcctaaggatatcctgcggatcaccagaacccccgaggtcacctgtgtcgtcctggatctgggccgggaagatcctgaagtgcagattagctggtttgtggacggcaaggaagtgcacacagctaagacccaatcccgggagcagcagttcaatggcacctaccgggtggtctctgtcctgcccatcgagcaccaagattggctgacaggcaaagagtttaagtgccgagtcaaccacatagatcttccctcccctattgagcggaccatctccaaggcacgggggcgagcgcacaaaccctctgtctatgtgctgcctccctctcccaaagaattgagctctagcgatacagtgtcaatcacctgcctgatcaaggacttctacccccctgacattgatgttgaatggcaatcaaatgggcagcaagaaccagagagaaaacacagaatgacccctccacagctggatgaggacgggtcctactttctgtactctaaactttccgtggacaagagcagatggcagcagggagaccctttcacctgtgcggtcatgcacgagacactgcaaaaccactacacagatctgtccttgagccactcacctggcaagtgataaggccggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatgtaccgcatgcaactcctgtcttgcattgcactaagtcttgcacttgtcacaaacagtgacatccagatgacccagaccaccagcagcctgagcgccagcctgggcgacagagtgatcatcagctgtagcgcctcccaggacatcagcaactacctgaactggtatcagcagaaacccgacggcaccgtgaaggtgctgatctacttcaccagctccctgcacagcggcgtgcccagcagattttctggcagcggctccggcaccgactacagcctgaccatctccaacctggaacccgaggatatcgccacctactactgccagcagtacagcaccgtgccctggacctttggcggaggcaccaagctggaaatcaagcggaatgatgctcagcctgctgtgtacctttttcaaccaagccctgaccaactgcataccggcagtgcctctgtggtctgcctgcttaatagcttctatcccaaggacattaatgtgaagtggaaggttgacggcgtgatacaggataccggaattcaggaaagtgtgacagaacaagataaggatagcacctatagcctgtctagcaccctcaccatgagcagcacagagtacttgagtcatgagctgtatagctgtgagattacccacaagagtctgccaagcacccttataaaaagtttccagcgatctgagtgttgatgaaagcttgcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcggggactttgcactggaacttacaacacccgagcaaggacgcgact

SEQ ID NO: 11 - DNA, 7584 bp, Artificial Sequence
  Constructed sequence
ctggcaagagaaagcggagagcccccgtgaagcagaccctgaacttcgacctgctgaagctggccggcgacgtggaaagcaaccctggccctatgtacaagatgcaactcctgtcttgcattgcactaactcttgtccttgtcgcaaacagtgacatccagatgacccagaccaccagcagcctgagcgccagcctgggcgacagagtgatcatcagctgtagcgcctcccaggacatcagcaactacctgaactggtatcagcagaaacccgacggcaccgtgaaggtgctgatctacttcaccagctccctgcacagcggcgtgcccagcagattttctggcagcggctccggcaccgactacagcctgaccatctccaacctggaacccgaggatatcgccacctactactgccagcagtacagcaccgtgccctggacctttggcggaggcaccaagctggaaatcaagcggaatgatgctcagcctgctgtgtacctttttcaaccaagccctgaccaactgcataccggcagtgcctctgtggtctgcctgcttaatagcttctatcccaaggacattaatgtgaagtggaaggttgacggcgtgatacaggataccgggattcaggaaagtgtgacagaacaagataaggatagcacctatagcctgtctagcaccctcaccatgagcagcacagagtacttgagtcatgagctgtatagctgtgagattacccacaagagtctgccaagcacccttataaaaagtttccagcgatctgagtgttgataaggtaccgcttgcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcggggactttgcactggaacttacaacacccgagcaaggccaccatgtacaagatgcaactcctgtcttgcattgcactaactcttgtccttgtcgcaaacagtgagatccagctggtgcagtctggccccgagctgaagcagcctggcgagacagtgcggatcagctgcaaggccagcggctacaccttcaccaactacggcatgaactgggtcaagcaggcccctggcaagggcctgaagtggatgggctggatcaacacctacaccggcgagcctacctacgccgccgacttcaagcggcggttcaccttcagcctggaaaccagcgccagcaccgcctacctgcagatcagcaacctgaagaacgacgacaccgccacctacttttgcgccaagtacccccactactacggcagcagccactggtacttcgacgtgtggggagccggcaccaccgtgacagtgtcatctgcgtcgaccacagccccctctgtgttccccctggccccttcctgtgggtcaacctctggcagcacagtggccctggcgtgtcttgtgtctggctacttccctgaacctgtgacagtcagctggaacagcggaagcctgacctctggagtgcacaccttccccagtgtcctgcaaagctcaggcctgcacagcctgtcaagtatggtgacagtgcccagtagcaggtggccttctgaaacctttacctgcaacgtggtgcaccctgcatccaacaccaaagtggataagcctgttttcaatgagtgcagatgcacagatacacctccctgccctgtgcctgagcctctgggaggaccatcagtcctgatcttccctccaaagcctaaggatatcctgcggatcaccagaacccccgaggtcacctgtgtcgtcctggatctgggccgggaagatcctgaagtgcagattagctggtttgtggacggcaaggaagtgcacacagctaagacccaatcccgggagcagcagttcaatggcacctaccgggtggtctctgtcctgcccatcgagcaccaagattggctgacaggcaaagagtttaagtgccgagtcaaccacatagatcttccctcccctattgagcggaccatctccaaggcacgggggcgagcgcacaaaccctctgtctatgtgctgcctccctctcccaaagaattgagctctagcgatacagtgtcaatcacctgcctgatcaaggacttctacccccctgacattgatgttgaatggcaatcaaatgggcagcaagaaccagagagaaaacacagaatgacccctccacagctggatgaggacgggtcctactttctgtactctaaactttccgtggacaagagcagatggcagcagggagaccctttcacctgtgcggtcatgcacgagacactgcaaaaccactacacagatctgtccttgagccactcac

SEQ ID NO: 12 - DNA, 7584 bp, Artificial Sequence; identical to SEQ ID NO: 11
  Constructed sequence

SEQ ID NO: 13 - DNA, 7241 bp, Artificial Sequence
  Constructed sequence
ccccgcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgccaccatgtacaagatgcaactcctgtcttgcattgcactaactcttgtccttgtcgcaaacagtgacatccagatgacccagaccaccagcagcctgagcgccagcctgggcgacagagtgatcatcagctgtagcgcctcccaggacatcagcaactacctgaactggtatcagcagaaacccgacggcaccgtgaaggtgctgatctacttcaccagctccctgcacagcggcgtgcccagcagattttctggcagcggctccggcaccgactacagcctgaccatctccaacctggaacccgaggatatcgccacctactactgccagcagtacagcaccgtgccctggacctttggcggaggcaccaagctggaaatcaagcggaatgatgctcagcctgctgtgtacctttttcaaccaagccctgaccaactgcataccggcagtgcctctgtggtctgcctgcttaatagcttctatcccaaggacattaatgtgaagtggaaggttgacggcgtgatacaggataccgggattcaggaaagtgtgacagaacaagataaggatagcacctatagcctgtctagcaccctcaccatgagcagcacagagtacttgagtcatgagctgtatagctgtgagattacccacaagagtctgccaagcacccttataaaaagtttccagcgatctgagtgttgataagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatgtacaagatgcaactcctgtcttgcattgcactaactcttgtccttgtcgcaaacagtgagatccagctggtgcagtctggccccgagctgaagcagcctggcgagacagtgcggatcagctgcaaggccagcggctacaccttcaccaactacggcatgaactgggtcaagcaggcccctggcaagggcctgaagtggatgggctggatcaacacctacaccggcgagcctacctacgccgccgacttcaagcggcggttcaccttcagcctggaaaccagcgccagcaccgcctacctgcagatcagcaacctgaagaacgacgacaccgccacctacttttgcgccaagtacccccactactacggcagcagccactggtacttcgacgtgtggggagccggcaccaccgtgacagtgtcatctgcgtcgaccacagccccctctgtgttccccctggccccttcctgtgggtcaacctctggcagcacagtggccctggcgtgtcttgtgtctggctacttccctgaacctgtgacagtcagctggaacagcggaagcctgacctctggagtgcacaccttccccagtgtcctgcaaagctcaggcctgcacagcctgtcaagtatggtgacagtgcccagtagcaggtggccttctgaaacctttacctgcaacgtggtgcaccctgcatccaacaccaaagtggataagcctgttttcaatgagtgcagatgcacagatacacctccctgccctgtgcctgagcctctgggaggaccatcagtcctgatcttccctccaaagcctaaggatatcctgcggatcaccagaacccccgaggtcacctgtgtcgtcctggatctgggccgggaagatcctgaagtgcagattagctggtttgtggacggcaaggaagtgcacacagctaagacccaatcccgggagcagcagttcaatggcacctaccgggtggtctctgtcctgcccatcgagcaccaagattggctgacaggcaaagagtttaagtgccgagtcaaccacatagatcttccctcccctattgagcggaccatctccaaggcacgggggcgagcgcacaaaccctctgtctatgtgctgcctccctctcccaaagaattgagctctagcgatacagtgtcaatcacctgcctgatcaaggacttctacccccctgacattgatgttgaatggcaatcaaatgggcagcaagaaccagagagaaaacacagaatgacccctccacagctggatgaggacgggtcctactttctgtactctaaactttccgtggacaagagcagatggcagcagggagaccctttcacctgtgcggtcatgcacgagacactgcaaaaccactacacagatctgtccttgagccactcacctggcaagtgataaggccgg